(19) United States
(12) Patent Application Publication
(10) Pub. No.: US 2024/0398478 A1
(43) Pub. Date: Dec. 5, 2024

(54) COMPUTER-ASSISTED SURGICAL METHOD FOR OSTEOTOMY

(71) Applicant: OSTESYS, Saint-Martin-D'Hères (FR)

(72) Inventors: Marie-Anne CHANRION, SAINT-MARTIN-D'HÈRES (FR); Teddy CHOPARD, SAINT-MARTIN-D'HÈRES (FR); Gilliane COSTA, SAINT-MARTIN-D'HÈRES (FR); Maeva ARFUSO, SAINT-MARTIN-D'HÈRES (FR)

(21) Appl. No.: 18/205,118

(22) Filed: Jun. 2, 2023

(51) Int. Cl. A61B 34/10; A61B 17/86; A61B 34/20

(52) U.S. Cl. CPC A61B 34/10; A61B 17/86; A61B 34/20; A61B 2017/681; A61B 2034/104; A61B 2034/105; A61B 2034/2051; A61B 2034/2065

(57) ABSTRACT

A computer-assisted surgical method for osteotomy of a first bone of a lower limb to correct a misalignment of the lower limb by moving a first and a second bone portion of the first bone away or toward each other, wherein a first tracker is fixed to the first bone portion and a second tracker to the second bone portion, the method comprising:
drilling a plurality of screw holes in the first bone;
performing at least one partial cut in the first bone to create the first and second bone portions;
wherein a third tracker is fixed to the second bone portion at a predetermined pose before or after drilling the plurality of screw holes or after performing the at least one partial cut,
the predetermined pose being determined from:
   a 3D model of the first bone from tracked poses of the first and second trackers; and
   a 3D forbidden region.

### TECHNICAL FIELD

The present subject matter relates generally to orthopedic surgery more particularly, to osteotomies.

### BACKGROUND

A limb of a human body comprises a first bone and a second bone articulated with the first bone. For example, the limb may be a leg wherein one of the first and second bones is a femur adjacent to a hip and the other one of the first and second bones is a tibia articulated with the femur by a knee and adjacent to an ankle.

Misalignment of the limb can cause osteoarthritis, or degradation of articular cartilage, resulting in pain, stiffness, and swelling. Osteotomy is a surgical procedure that may be performed on the leg and is used to correct such misalignment to relieve pressure on a portion of the joint by shifting the load-bearing axis.

To that end, some osteotomy procedures may comprise cutting a part of a bone of said limb to create a bone hinge and moving the bone portions around the hinge to achieve a better alignment of the limb. Once said alignment has been achieved, an implant is placed between the bone portions and screwed onto each bone portion to maintain them in said corrected pose.

However, osteotomies are complex and require not only a precise diagnosis of the misalignment but also an accurate control of the surgical procedure itself.

Bone portions may be tracked while the surgeon moves bone portions to improve control accuracy.

However, tracking both bone portions may raise issues not present, or less prevalent, when the bone is tracked as a whole. For example, a tracker on the bone may interfere with surgical components, such as an implant to maintain the bone in the deformed configuration, or may be affected by vibrations caused by surgical operations, such as bone cutting.

Accordingly, improving tracker posing in bone portions that help overcome these issues would be useful.

### SUMMARY

According to a first aspect of the present disclosure, it is provided a computer-assisted surgical method for osteotomy of a first bone of a lower limb to correct a misalignment of the lower limb by moving a first bone portion of the first bone and a second bone portion of the first bone away from or toward each other about a bony hinge to reach a targeted deformed configuration of the first bone achieving a corrected alignment of the lower limb, the first bone portion and the second bone portion being created after cutting the first bone, the lower limb comprising the first bone and a second bone articulated with the first bone, the method comprising:
fixing a first tracker to the first bone and a second tracker to the second bone;
based on tracked poses of the first tracker and of the second tracker, computing the targeted deformed configuration of the first bone achieving the corrected alignment of the lower limb, at least one cutting plane and an optimal placement of an implant to maintain the first bone in the targeted deformed configuration;
fixing a third tracker to the second bone portion of the first bone to a predetermined pose;
after fixing the third tracker, drilling a plurality of screw holes in the first bone on either side of the at least one computed cutting plane;
after drilling the plurality of screw holes, performing at least one partial cut in the first bone along the at least one computed cutting plane to create the first bone portion and the second bone portion;
based on tracked poses of the first, second and third trackers, moving the first bone portion and the second bone portion away or toward each other to reach the targeted deformed configuration; and
fixing the implant in the optimal placement to the first bone portion and the second bone portion with a plurality of screws in the plurality of respective screw holes to maintain the first bone in the targeted deformed configuration,
   wherein the method comprises:
determining the predetermined pose of the third tracker on the second bone portion of the first bone from:
   a 3D model of the first bone from tracked poses of the first tracker and the second tracker; and
   a 3D forbidden region comprising at least one of:
      a first 3D region around the at least one computed cutting plane, wherein the at least one partial cut in the first bone is performed along the at least one computed cutting plane to create the first bone portion and the second bone portion;
      a second 3D region representing a volume of the implant at the optimal placement to maintain the first bone in the targeted deformed configuration;
      a third 3D region representing a volume of the plurality of screws in the plurality of respective screw holes;
      a 3D anatomical region of the first bone, wherein the 3D anatomical region comprises:
         a 3D joint region adjacent to the second bone portion of the first bone; and
         a 3D vascular region of the first bone,
wherein the predetermined pose is outside the 3D forbidden region.

By “pose”, we mean both positions and orientations (i.e. six degrees of freedom in all) of an object, such as a tracker.

The computer-assisted surgical method according to the first aspect may further comprise displaying the 3D model of the first bone and the 3D forbidden region.

The computer-assisted surgical method according to the first aspect may further comprise displaying the first portion and the second portion in the 3D model.

The first tracker, the second tracker, and the third tracker according to the first aspect may be respectively configured to receive an electromagnetic field, the electromagnetic field being emitted by an emitting device, and tracked poses of the first tracker, the second tracker, and the third tracker according to the first aspect may be computed based on the received electromagnetic field.

The computer-assisted surgical method according to the first aspect may further comprise:
obtaining a 2D image or a 3D image comprising:
   the first bone;
   the implant;
   the plurality of screws; and
   the plurality of respective screw holes; and
controlling from the 2D image or from the 3D image the pose of the implant and the pose of the plurality of screws.

According to a second aspect of the present disclosure, it is provided a computer-assisted surgical method for osteotomy of a first bone of a lower limb to correct a misalignment of the lower limb by moving a first bone portion of the first bone and a second bone portion of the first bone away from or toward each other about a bony hinge to reach a targeted deformed configuration of the first bone achieving a corrected alignment of the lower limb, the first bone portion and the second bone portion being created after cutting the first bone, the lower limb comprising the first bone and a second bone articulated with the first bone, the method comprising:
fixing a first tracker to the first bone and a second tracker to the second bone;
based on tracked poses of the first tracker and the second tracker, computing the targeted deformed configuration of the first bone achieving the corrected alignment of the lower limb, at least one cutting plane and an optimal placement of an implant to maintain the first bone in the targeted deformed configuration;
drilling a plurality of screw holes in the first bone on either side of the at least one computed cutting plane;
after drilling the plurality of screw holes, fixing a third tracker to the second bone portion of the first bone to a predetermined pose;
after fixing the third tracker, performing at least one partial cut in the first bone along the at least one computed cutting plane to create the first bone portion and the second bone portion;
based on tracked poses of the first, second and third trackers, moving the first bone portion and the second bone portion away or toward each other to reach the targeted deformed configuration; and
fixing the implant in the optimal placement to the first bone portion and the second bone portion with a plurality of screws in the plurality of respective screw holes to maintain the first bone in the targeted deformed configuration,
   wherein the method comprises:
determining the predetermined pose of the third tracker on the second portion of the first bone from:
   a 3D model of the first bone from tracked poses of the first tracker and the second tracker; and
   a 3D forbidden region comprising at least one of:
      a first 3D region around the at least one computed cutting plane, wherein the at least one partial cut in the first bone is performed along the at least one computed cutting plane to create the first bone portion and the second bone portion;
      a second 3D region representing a volume of the implant at the optimal placement to maintain the first bone in the targeted deformed configuration;
      a third 3D region representing a volume of the plurality of screws in the plurality of respective screw holes;
      a 3D anatomical region of the first bone, wherein the 3D anatomical region comprises:
         a 3D joint region adjacent to the second bone portion; and
         a 3D vascular region of the first bone,
            wherein the predetermined pose is outside the 3D forbidden region.

The computer-assisted surgical method according to the second aspect may further comprise displaying the 3D model of the first bone and the 3D forbidden region.

The computer-assisted surgical method according to the second aspect, may further comprise displaying the first portion and the second portion in the 3D model.

The first tracker, the second tracker, and the third tracker according to the second aspect may be respectively configured to receive an electromagnetic field, the electromagnetic field being emitted by an emitting device, and tracked poses of the first tracker, the second tracker, and the third tracker according to the second aspect may be computed based on the received electromagnetic field.

The computer-assisted surgical method according to the second aspect may further comprise:
obtaining a 2D image or a 3D image comprising:
   the first bone;
   the implant;
   the plurality of screws; and
   the plurality of respective screw holes; and
controlling from the 2D image or from the 3D image the pose of the implant and the pose of the plurality of screws.

According to a third aspect of the present disclosure, it is provided a computer-assisted surgical method for osteotomy of a first bone of a lower limb to correct a misalignment of the lower limb by moving a first bone portion of the first bone and a second bone portion of the first bone away from or toward each other about a bony hinge to reach a targeted deformed configuration of the first bone achieving a corrected alignment of the lower limb, the first bone portion and the second bone portion being created after cutting the first bone, the lower limb comprising the first bone and a second bone articulated with the first bone, the method comprising:
fixing a first tracker to the first bone and a second tracker to the second bone;
based on tracked poses of the first tracker and the second tracker, computing the targeted deformed configuration of the first bone achieving the corrected alignment of the lower limb, at least one cutting plane and an optimal placement of an implant to maintain the first bone in the targeted deformed configuration;
drilling a plurality of screw holes in the first bone on either side of the at least one computed cutting plane;
after drilling the plurality of screw holes, performing at least one partial cut in the first bone along the at least one computed cutting plane to create the first bone portion and the second bone portion;
after performing the at least one partial cut, fixing a third tracker to the second bone portion of the first bone to a predetermined pose;
based on tracked poses of the first, second and third trackers, moving the first bone portion and second bone portion away or toward each other to reach the targeted deformed configuration; and
fixing the implant in the optimal placement to the first bone portion and the second bone portion with a plurality of screws in the plurality of respective screw holes to maintain the first bone in the targeted deformed configuration,
   wherein the method comprises:
determining the predetermined pose of the third tracker on the second portion of the first bone from:
   a 3D model of the first bone from tracked poses of the first tracker and the second tracker; and
   a 3D forbidden region comprising at least one of:
      a first 3D region around the at least one computed cutting plane, wherein the at least one partial cut in the first bone is performed along the at least one computed cutting plane to create the first bone portion and the second bone portion;
      a second 3D region representing a volume of the implant at the optimal placement to maintain the first bone in the targeted deformed configuration;
      a third 3D region representing a volume of the plurality of screws in the plurality of respective screw holes;
      a 3D anatomical region of the first bone, wherein the 3D anatomical region comprises:
         a 3D joint region adjacent to the second bone portion of the first bone; and
         a 3D vascular region of the first bone,
wherein the predetermined pose is outside the 3D forbidden region.

The computer-assisted surgical method according to the third aspect may further comprise displaying the 3D model of the first bone and the 3D forbidden region.

The computer-assisted surgical method according to the third aspect, may further comprise displaying the first portion and the second portion in the 3D model.

The first tracker, the second tracker, and the third tracker according to the third aspect may be respectively configured to receive an electromagnetic field, the electromagnetic field being emitted by an emitting device, and tracked poses of the first tracker, the second tracker, and the third tracker according to the third aspect may be computed based on the received electromagnetic field.

The computer-assisted surgical method according to the third aspect may further comprise:
obtaining a 2D image or a 3D image comprising:
   the first bone;
   the implant;
   the plurality of screws; and
   the plurality of respective screw holes; and
controlling from the 2D image or from the 3D image the pose of the implant and the pose of the plurality of screws.

### BRIEF DESCRIPTION OF THE DRAWINGS

FIG. 1a to 1d are schematic views of a first bone and a second bone of a limb showing a pose of a first tracker, a second tracker and a third tracker for respectively a high-tibial osteotomy (FIG. 1a), a distal tibial osteotomy (FIG. 1b), a distal femoral osteotomy (FIG. 1c) and a proximal femoral osteotomy (FIG. 1d) in accordance with an exemplary embodiment of the present subject matter.

FIG. 2 is a schematic view of the second bone comprising a distractor for moving a first bone portion and a second bone portion away from or toward each other to reach a targeted deformed configuration in accordance with an exemplary embodiment of the present subject matter.

FIG. 3a to 3c are schematic views of the first bone, a bony hinge, an implant, a plurality of screws and the third tracker.

FIG. 4 is a schematic view of the first bone, a cutting plane and a 3D forbidden region in accordance with an exemplary embodiment of the present subject matter.

FIG. 5 illustrates, in case of a high-tibial osteotomy, a schematic view of the first bone, the bony hinge, the implant, the plurality of screws and the third tracker at a predetermined pose outside the 3D forbidden region in accordance with an exemplary embodiment of the present subject matter.

FIG. 6 is a flow chart representing several steps of a computer-assisted method in accordance with a first exemplary embodiment of the present subject matter.

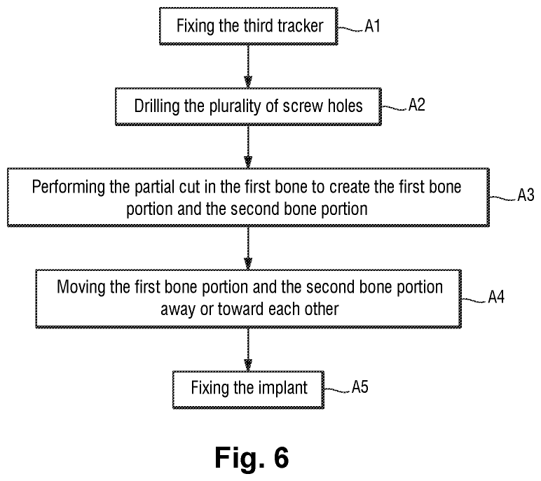

FIG. 7 is a flow chart representing several steps of the computer-assisted method in accordance with a second exemplary embodiment of the present subject matter.

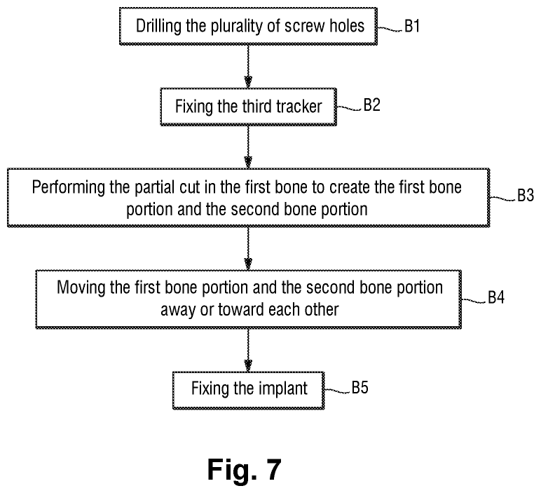

FIG. 8 is a flow chart representing several steps of the computer-assisted method in accordance with a third exemplary embodiment of the present subject matter.

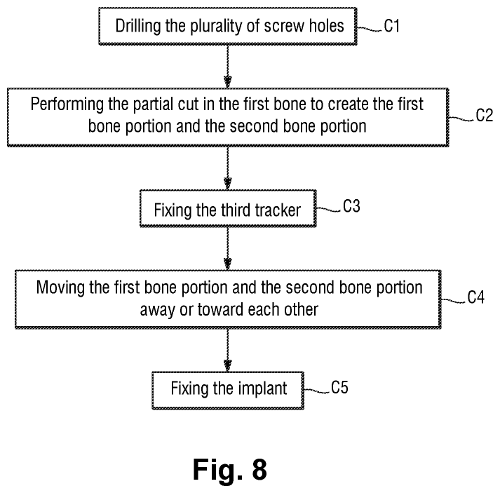

### DETAILED DESCRIPTION

An osteotomy of a first 1, respectively second bone 2 of a lower limb is a surgical operation wherein the first 1, respectively second bone 2 is cut and reconfigured to correct a misaligned in the lower limb.

In the following description, it is assumed for the sake of conciseness that osteotomy is carried out on the first bone 1, but osteotomy may, in addition or alternatively, be carried out on the second bone 2.

In an open wedge osteotomy, a partial cut is made through the first bone 1 to obtain a first bone portion 11 and a second bone portion 12. The first and the second bone portions 11, 12 can then be moved away from each other about a bony hinge 13 to reach an opened targeted deformed configuration of the first bone 1 achieving a corrected alignment of the limb. In a close wedge osteotomy, partial cuts are performed in the first bone 1 to remove a wedge-shaped portion to obtain the first bone portion 11 and the second bone portion 12 that are moved toward each other about the bony hinge 13 to reach a closed targeted deformed configuration of the first bone 1 achieving a corrected alignment of the lower limb.

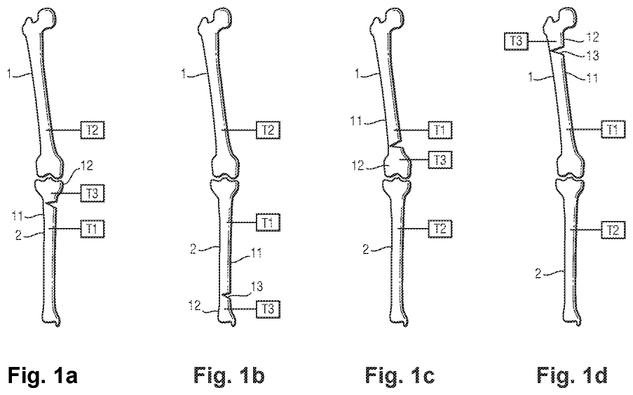

FIG. 1a to 1d illustrate schematically different poses wherein each partial cut may be performed for a closed or opened wedge osteotomy. For example, the cut may be performed in the tibia, adjacent to the knee, in case of a high-tibial osteotomy as illustrated in FIG. 1a, or adjacent to the ankle, in case of a distal tibial osteotomy as illustrated in FIG. 1b. The cut may also be performed in the femur, adjacent to the knee, in case of a distal femoral osteotomy illustrated in FIG. 1c, or adjacent the hip, in case of a proximal femoral osteotomy as illustrated in FIG. 1d.

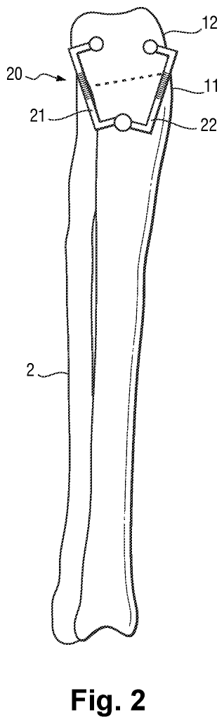

In one embodiment, a tool may be used to move the first bone portion and the second bone portion. Said tool may be a distractor 20 fixed to the first bone 1 and comprising telescopic arms 21, 22 configured to move the first and the second bone portions 21, 22 away from each other as illustrated in FIG. 2.

An implant 60 is then fixed to the first bone 1 with a plurality of screws 70 in a plurality of respective screw holes previously drilled in the first bone 1 to maintain the first bone 1 in the deformed configuration.

To improve surgical planning of the above osteotomies, a control unit is configured to compute a 3D model of the first bone based on data provided by an electromagnetic tracking system, 3D images obtained for example by CT scan or intra-operative CBCT and/or measurements obtained by palpation of the outer surface of the first and/or second bone by the surgeon.

The control unit is also configured to compute, based on data provided by the electromagnetic tracking system, a targeted deformed configuration of the first bone 1 achieving the corrected alignment of the lower limb, at least one cutting plane 50, an optimal placement of the implant 60 to maintain the first bone 1 in the targeted deformed configuration and a model of the plurality of screws 70.

The control unit may, for instance, comprise one or more microprocessor, one or more random access memory (RAM) and/or one or more read-only memory (ROM), one or more calculators, one or more computers and/or one or more computer programs. The computer program(s) comprise code instructions to compute the 3D model, the targeted deformed configuration, the at least one cutting plane 50, the optimal placement of the implant 60 and the model of the plurality of screws 70. In addition, the control unit may include other devices and circuitry for performing the functions described herein such as, for example, a hard drive, input/output circuitry, and the like. The input/output circuitry can be adapted to treat digital and/or analog signals emitted from the electromagnetic tracking system.

The electromagnetic tracking system comprises a first tracker T1 and a second tracker T2, each of the first and second trackers T1, T2 being configured to receive an electromagnetic field, and an emitting device configured to emit the electromagnetic field, the control unit being configured to determine, from the electromagnetic field received by the first and second trackers T1, T2, a pose of the first tracker T1 with respect to the emitting device, a pose of the second tracker T2 with respect to the emitting device, and a pose of the first tracker T1 with respect to the second tracker T2 based on the determined pose of the first and second trackers T1, T2 with respect to the emitting device.

In one embodiment, each of the first tracker T1 and the second tracker T2 comprises receiving coils, for example three receiving coils or more, each receiving coil being configured to receive a component of the electromagnetic field, and the emitting device comprises three emitting coils, each emitting coil being configured to emit the component of the electromagnetic field received by the respective receiving coil.

The first tracker T1 is fixed to the first bone 1 and the second tracker T2 is fixed to the second bone 2, for example by impacting the first tracker T1 and the second tracker T2 into a respective pose in the first bone 1 and in the second bone 2. Some examples of poses of the first tracker T1 and of the second tracker T2 are illustrated in FIGS. 1a to 1d.

In one embodiment, the 3D model of the first bone 1 further comprises the first bone portion 11 and the second bone portion 12.

In one embodiment, the 3D model of the first bone 1 is displayed in a 3D image, for example by a screen configured to receive data from the control unit, and the surgeon may navigate in a 3D space associated with the 3D image thanks to the electromagnetic tracking system.

In another embodiment, the targeted deformed configuration, the at least one cutting plane 50, the optimal placement of the implant 60 and the pose of the plurality of screws 70 are displayed in the 3D image.

Once the surgeon partially cuts the first bone 1 to form the first and second bone portions 11, 12, the electromagnetic tracking system is no longer able to determine a pose of the second bone portion 12 (on the non-tracked side of the cut) with respect to the first bone portion 11 when the first and second bone portions 11, 12 are moved relative to each other.

Consequently, the electromagnetic tracking system further comprises a third tracker T3 fixed to the second bone portion 12, for example by impacting the third tracker T3 into a respective pose in the first bone 1.

The third tracker T3 is generally similar to the first and second trackers T1, T2. In one embodiment, the third tracker T3 comprises receiving coils, for example three receiving coils or more, each receiving coil being configured to receive the component of the electromagnetic field.

The third tracker T3 being configured to receive the electromagnetic field, the control unit is configured to determine, from the electromagnetic field received by the third tracker T3, a pose of the third tracker T3 with respect to the emitting device. The control unit is also configured to determine, based on the pose of the first, second and third trackers T1, T2, T3 with respect to the emitting device, a pose of the third tracker T3 with respect to the first tracker T1, and a pose of the third tracker T3 with respect to the second tracker T2. Some examples of poses of the third tracker T3 are illustrated in FIGS. 1a to 1d.

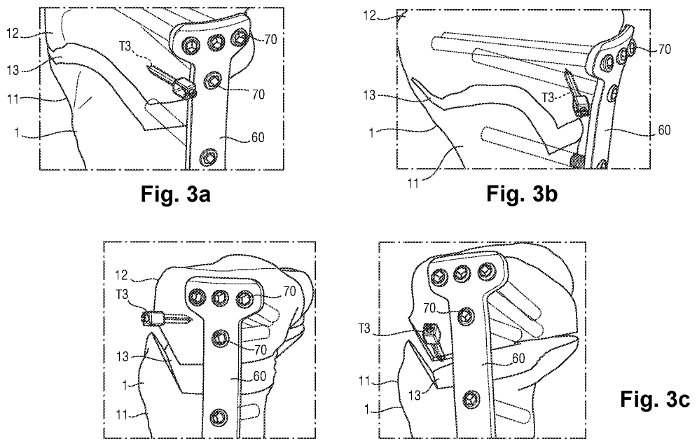

However, it remains difficult for the surgeon to pose the third tracker T3 in the second bone portion 12. Indeed, the third tracker T3 may obstruct drilling and cutting operations when they are performed by the surgeon, as illustrated in FIGS. 3b and 3c wherein the third tracker T3 is inside a screw hole and inside the bony hinge 13.

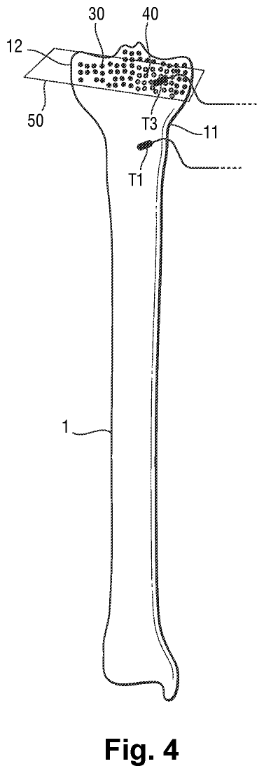

Also, the third tracker T3 may disturb the surgeon when fixing the implant 60 because the third tracker T3 may be in a pose shared with the implant 60 and the plurality of screws 70 as illustrated in FIGS. 3a and 3b showing the third tracker T3 superimposed with the implant 60 (FIG. 4a) and superimposed with the plurality of screws 70 (FIG. 3b).

Therefore, a predetermined pose of the third tracker T3 on the second bone portion 12 of the first bone 1 is advantageously computed, for example by the control unit, from the 3D model of the first bone 1 obtained from tracked poses of the first tracker T1 and the second tracker T2, and from a 3D forbidden region 30, the predetermined pose of the third tracker T3 being outside the 3D forbidden region 30.

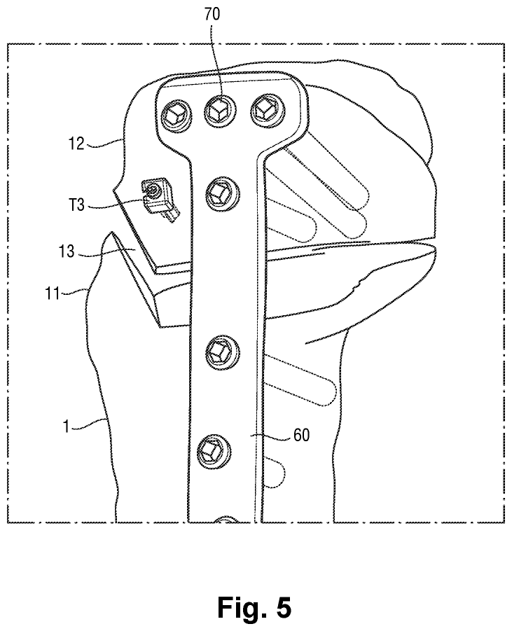

Defining the predetermined pose of the third tracker T3 allows the surgeon to fix the third tracker T3 properly to perform osteotomy without interfering with material and surgery. For instance, as shown in FIG. 5 in case of a high-tibial osteotomy, the third tracker T3 is distant from the implant 60 and the plurality of screws 70. The third tracker T3 is also distant from the bony hinge 13 in order to allow the surgeon to perform partial cuts and, in case of close wedge osteotomy, to remove the wedge-shaped portion. Moreover, the third tracker T3 is distant from anatomical regions, such as the knee (in the case of the high-tibial osteotomy), in order to avoid joint regions of the lower limb, and vascular areas.

The 3D forbidden region 30 may thus be formed of one or several 3D region(s) (i.e. volumes) that may be distant from each other or at least partially overlap. The 3D forbidden region may comprise at least one of:
a first 3D region around the at least one computed cutting plane to allow the surgeon to perform the corresponding partial cut;
a second 3D region, to avoid the implant 60, representing a volume of the implant 60 at the optimal placement; and
a third 3D region, to avoid the plurality of screws 70, representing a volume of the plurality of screws 70 in the plurality of respective screw holes.

The size of the first 3D region is bigger in case of the close wedge osteotomy than in case of the open wedge osteotomy. Indeed, open wedge osteotomy needs one partial cut and the first 3D region is around this partial cut. However, close wedge osteotomy needs at least two partial cuts and a wedge-shaped portion is removed from the first bone 1. The 3D region extends therefore around these partial cuts and the removed portion of the first bone 1. In addition, the volume of the implant at the optimal placement comprises the area where the implant is implanted and also the volume occupied by the implant 60.

In one embodiment, the first, second and/or third 3D regions are obtained by the control unit based on the targeted deformed configuration of the first bone 1, the at least one cutting plane 50, the optimal placement of the implant 60 and the model of the plurality of screws 70.

In order to avoid anatomical regions, such as joint regions of the limb and vascular areas, the 3D anatomical region of the first bone may also comprise:
a 3D joint region adjacent to the second bone portion 12 of the first bone 1, such as tibial tray, talus, and condyles; and
a 3D vascular region of the first bone 1, for example a region where an artery passes behind the knee.

In one embodiment, the 3D joint region and/or the 3D vascular region are obtained by the control unit based on the 3D model of the first bone 1 and on known anatomical features of the lower limb.

The 3D forbidden region 30 may be displayed and be superimposed to the 3D image of the first bone 1. For example, FIG. 4 illustrates a schematic displayed view comprising the first bone 1 and a cutting plane 50 delimiting the first bone portion 11 and the second bone portion 12, corresponding to the 3D image, and the 3D forbidden region 30 (represented by dots in FIG. 4) to avoid. Another region 40 (represented by rings in FIG. 4) shows the possible poses to fix the third tracker T3 without interfering with surgical steps.

Therefore, correctly posing the third tracker T3 thanks to the 3D forbidden area allows the surgeon to fix the third tracker T3 in the first bone 1 in a pose which does not interfere while performing osteotomy.

### Examples of Third Tracker T3 Fixation During Osteotomy

The third tracker T3 may be fixed at the predetermined pose at different moments during osteotomy.

In a first embodiment, the third tracker T3 is fixed, during a first step A1, to the second bone portion 12 of the first bone 1 to the predetermined pose;
after fixing the third tracker T3, a plurality of screw holes are drilled (step A2) in the first bone 1 on either side of the at least one computed cutting plane 50;
after drilling the plurality of screw holes, at least one partial cut is performed (step A3) in the first bone 1 along the at least one computed cutting plane 50 to create the first bone portion 11 and the second bone portion 12;
based on tracked poses of the first, second and third trackers, T1, T2, T3 the first bone portion 11 and the second bone portion 12 are moved (step A4) away or toward each other to reach the targeted deformed configuration; and
the implant 60 is fixed (step A5) in the optimal placement to the first bone portion 11 and the second bone portion 12 with a plurality of screws 70 in the plurality of respective screw holes to maintain the first bone 1 in the targeted deformed configuration.

Thanks to the predetermined pose, the third tracker T3 (fixed at step A1) does not interfere with surgical operations, such as drilling and cutting, with the implant or with the plurality of screws.

In a second embodiment, a plurality of screw holes is drilled, during a first step B1, in the first bone 1 on either side of the at least one computed cutting plane 50; after drilling the plurality of screw holes, the third tracker T3 is fixed (step B2) to the second bone portion 12 of the first bone 1 to the predetermined pose;
after fixing the third tracker T3, at least one partial cut is performed (step B3) in the first bone 1 along the at least one computed cutting plane 50 to create the first bone portion 11 and the second bone portion 12;
based on tracked poses of the first, second and third trackers, T1, T2, T3 the first bone portion 11 and the second bone portion 12 are moved (step B4) away or toward each other to reach the targeted deformed configuration; and
the implant 60 is fixed (step B5), according to the optimal placement, to the first bone portion 11 and the second bone portion 12 with a plurality of screws in the plurality of respective screw holes to maintain the first bone 1 in the targeted deformed configuration.

Fixing the third tracker T3 at step B2 prevents the third tracker T3 from being subjected to vibrations induced while drilling the plurality of screw holes. Besides, since the third tracker is fixed before cutting the bone, an optimal accuracy of posing of the third tracker T3 can be achieved.

In a third embodiment, a plurality of screw holes is drilled during a first step C1, in the first bone 1 on either side of the at least one computed cutting plane 50;
after drilling the plurality of screw holes, at least one partial cut is performed (step C2) in the first bone 1 along the at least one computed cutting plane 50 to create the first bone portion 11 and the second bone portion 12;
after performing the at least one partial cut, the third tracker T3 is fixed (step C3) to the second bone portion 12 of the first bone 1 to the predetermined pose;
based on tracked poses of the first, second and third trackers, T1, T2, T3 the first bone portion 11 and second bone portion 12 are moved (step C4) away or toward each other to reach the targeted deformed configuration; and
the implant is fixed (step C5) in the optimal placement to the first bone portion 11 and the second bone portion 12 with a plurality of screws 70 in the plurality of respective screw holes to maintain the first bone 1 in the targeted deformed configuration.

Fixing the third tracker T3 at step C3 further prevents the third tracker T3 from being subjected to vibrations induced while drilling the plurality of screw holes and furthermore while performing the at least one partial cut. Therefore, the third tracker T3 holds at the predetermined pose.

The implant 60 and the plurality of screws 70 may be controlled after fixing the implant to check if the targeted deformation is reached and/or if the implant 60 is accurately posed in the first bone 1. In one embodiment, to validate the osteotomy, the implant 60 and the plurality of screws 70 may be controlled from a 2D image or a 3D image previously obtained.

1. A computer-assisted surgical method for osteotomy of a first bone of a lower limb to correct a misalignment of the lower limb by moving a first bone portion of the first bone and a second bone portion of the first bone away from or toward each other about a bony hinge to reach a targeted deformed configuration of the first bone achieving a corrected alignment of the lower limb, the first bone portion and the second bone portion being created after cutting the first bone, the lower limb comprising the first bone and a second bone articulated with the first bone, the method comprising:
fixing a first tracker to the first bone and a second tracker to the second bone;
based on tracked poses of the first tracker and of the second tracker, computing the targeted deformed configuration of the first bone achieving the corrected alignment of the lower limb, at least one cutting plane and an optimal placement of an implant to maintain the first bone in the targeted deformed configuration;
fixing a third tracker to the second bone portion of the first bone to a predetermined pose;
after fixing the third tracker, drilling a plurality of screw holes in the first bone on either side of the at least one computed cutting plane;
after drilling the plurality of screw holes, performing at least one partial cut in the first bone along the at least one computed cutting plane to create the first bone portion and the second bone portion;
based on tracked poses of the first, second and third trackers, moving the first bone portion and the second bone portion away or toward each other to reach the targeted deformed configuration; and
fixing the implant in the optimal placement to the first bone portion and the second bone portion with a plurality of screws in the plurality of respective screw holes to maintain the first bone in the targeted deformed configuration,
wherein the method comprises:
determining the predetermined pose of the third tracker on the second bone portion of the first bone from:
a 3D model of the first bone from tracked poses of the first tracker and the second tracker; and
a 3D forbidden region comprising at least one of:
a first 3D region around the at least one computed cutting plane, wherein the at least one partial cut in the first bone is performed along the at least one computed cutting plane to create the first bone portion and the second bone portion;
a second 3D region representing a volume of the implant at the optimal placement to maintain the first bone in the targeted deformed configuration;
a third 3D region representing a volume of the plurality of screws in the plurality of respective screw holes;
a 3D anatomical region of the first bone, wherein the 3D anatomical region comprises:
a 3D joint region adjacent to the second bone portion of the first bone; and
a 3D vascular region of the first bone,
wherein the predetermined pose is outside the 3D forbidden region.

2. The computer-assisted surgical method of claim 1, further comprising displaying the 3D model of the first bone and the 3D forbidden region.

3. The computer-assisted surgical method of claim 1, further comprising displaying the first portion and the second portion in the 3D model.

4. The computer-assisted surgical method of claim 1, wherein the first tracker, the second tracker, and the third tracker are respectively configured to receive an electromagnetic field, the electromagnetic field being emitted by an emitting device, and tracked poses of the first tracker, the second tracker, and the third tracker are computed based on the received electromagnetic field.

5. The computer-assisted surgical method of claim 1, further comprising:
obtaining a 2D image or a 3D image comprising:
the first bone;
the implant;
the plurality of screws; and
the plurality of respective screw holes; and
controlling from the 2D image or from the 3D image the pose of the implant and the pose of the plurality of screws.

6. A computer-assisted surgical method for osteotomy of a first bone of a lower limb to correct a misalignment of the lower limb by moving a first bone portion of the first bone and a second bone portion of the first bone away from or toward each other about a bony hinge to reach a targeted deformed configuration of the first bone achieving a corrected alignment of the lower limb, the first bone portion and the second bone portion being created after cutting the first bone, the lower limb comprising the first bone and a second bone articulated with the first bone, the method comprising:
fixing a first tracker to the first bone and a second tracker to the second bone;
based on tracked poses of the first tracker and the second tracker, computing the targeted deformed configuration of the first bone achieving the corrected alignment of the lower limb, at least one cutting plane and an optimal placement of an implant to maintain the first bone in the targeted deformed configuration;
drilling a plurality of screw holes in the first bone on either side of the at least one computed cutting plane;
after drilling the plurality of screw holes, fixing a third tracker to the second bone portion of the first bone to a predetermined pose;
after fixing the third tracker, performing at least one partial cut in the first bone along the at least one computed cutting plane to create the first bone portion and the second bone portion;
based on tracked poses of the first, second and third trackers, moving the first bone portion and the second bone portion away or toward each other to reach the targeted deformed configuration; and
fixing the implant in the optimal placement to the first bone portion and the second bone portion with a plurality of screws in the plurality of respective screw holes to maintain the first bone in the targeted deformed configuration,
wherein the method comprises:
determining the predetermined pose of the third tracker on the second portion of the first bone from:
a 3D model of the first bone from tracked poses of the first tracker and the second tracker; and
a 3D forbidden region comprising at least one of:
a first 3D region around the at least one computed cutting plane, wherein the at least one partial cut in the first bone is performed along the at least one computed cutting plane to create the first bone portion and the second bone portion;
a second 3D region representing a volume of the implant at the optimal placement to maintain the first bone in the targeted deformed configuration;
a third 3D region representing a volume of the plurality of screws in the plurality of respective screw holes;
a 3D anatomical region of the first bone, wherein the 3D anatomical region comprises:
a 3D joint region adjacent to the second bone portion; and
a 3D vascular region of the first bone,
wherein the predetermined pose is outside the 3D forbidden region.

7. The computer-assisted surgical method of claim 6, further comprising displaying the 3D model of the first bone and the 3D forbidden region.

8. The computer-assisted surgical method of claim 6, further comprising displaying the first portion and the second portion in the 3D model.

9. The computer-assisted surgical method of claim 6, wherein the first tracker, the second tracker, and the third tracker are respectively configured to receive an electromagnetic field, the electromagnetic field being emitted by an emitting device, and tracked poses of the first tracker, the second tracker, and the third tracker are computed based on the received electromagnetic field.

10. The computer-assisted surgical method of claim 6, further comprising:
obtaining a 2D image or a 3D image comprising:
the first bone;
the implant;
the plurality of screws; and
the plurality of respective screw holes; and
controlling from the 2D image or from the 3D image the pose of the implant and the pose of the plurality of screws.

11. A computer-assisted surgical method for osteotomy of a first bone of a lower limb to correct a misalignment of the lower limb by moving a first bone portion of the first bone and a second bone portion of the first bone away from or toward each other about a bony hinge to reach a targeted deformed configuration of the first bone achieving a corrected alignment of the lower limb, the first bone portion and the second bone portion being created after cutting the first bone, the lower limb comprising the first bone and a second bone articulated with the first bone, the method comprising:
fixing a first tracker to the first bone and a second tracker to the second bone;
based on tracked poses of the first tracker and the second tracker, computing the targeted deformed configuration of the first bone achieving the corrected alignment of the lower limb, at least one cutting plane and an optimal placement of an implant to maintain the first bone in the targeted deformed configuration;
drilling a plurality of screw holes in the first bone on either side of the at least one computed cutting plane;
after drilling the plurality of screw holes, performing at least one partial cut in the first bone along the at least one computed cutting plane to create the first bone portion and the second bone portion;
after performing the at least one partial cut, fixing a third tracker to the second bone portion of the first bone to a predetermined pose;
based on tracked poses of the first, second and third trackers, moving the first bone portion and second bone portion away or toward each other to reach the targeted deformed configuration; and
fixing the implant in the optimal placement to the first bone portion and the second bone portion with a plurality of screws in the plurality of respective screw holes to maintain the first bone in the targeted deformed configuration,
wherein the method comprises:
determining the predetermined pose of the third tracker on the second portion of the first bone from:
a 3D model of the first bone from tracked poses of the first tracker and the second tracker; and
a 3D forbidden region comprising at least one of:
a first 3D region around the at least one computed cutting plane, wherein the at least one partial cut in the first bone is performed along the at least one computed cutting plane to create the first bone portion and the second bone portion;
a second 3D region representing a volume of the implant at the optimal placement to maintain the first bone in the targeted deformed configuration;
a third 3D region representing a volume of the plurality of screws in the plurality of respective screw holes;
a 3D anatomical region of the first bone, wherein the 3D anatomical region comprises:
a 3D joint region adjacent to the second bone portion of the first bone; and
a 3D vascular region of the first bone,
wherein the predetermined pose is outside the 3D forbidden region.

12. The computer-assisted surgical method of claim 11, further comprising displaying the 3D model of the first bone and the 3D forbidden region.

13. The computer-assisted surgical method of claim 11, further comprising displaying the first portion and the second portion in the 3D model.

14. The computer-assisted surgical method of claim 11, wherein the first tracker, the second tracker, and the third tracker are respectively configured to receive an electromagnetic field, the electromagnetic field being emitted by an emitting device, and tracked poses of the first tracker, the second tracker, and the third tracker are computed based on the received electromagnetic field.

15. The computer-assisted surgical method of claim 11, further comprising:
obtaining a 2D image or a 3D image comprising:
the first bone;
the implant;
the plurality of screws; and
the plurality of respective screw holes; and
controlling from the 2D image or from the 3D image the pose of the implant and the pose of the plurality of screws.
